(19) United States
(12) Patent Application Publication
(10) Pub. No.: US 2007/0060830 A1
(43) Pub. Date: Mar. 15, 2007

(54) Method and system for detecting and classifying facial muscle movements

(76) Inventors: Tan Thi Thai Le, New South Wales (AU); Nam Hoai Do, New South Wales (AU); William Andrew King, New South Wales (AU); Hai Ha Pham, New South Wales (AU); Johnson Thie, New South Wales (AU); Emir Delic, New South Wales (AU)

Correspondence Address: FISH & RICHARDSON P.C., PO BOX 1022, MINNEAPOLIS, MN, 55440-1022, US

(21) Appl. No.: 11/225,598

(22) Filed: Sep. 12, 2005

(51) Int. Cl. A61B 5/04

(52) U.S. Cl. 600/544; 600/546

(57) ABSTRACT

A method of detecting and classifying facial muscle movements, comprising the steps of: detecting bio-signals from a plurality of scalp electrodes; and applying one or more than one facial muscle movement-detection algorithm to a portion of the bio-signals affected by a predefined type of facial muscle movement in order to detect facial muscle movements of that predefined type.

### FIELD

The present invention relates generally to the detection and classification of facial muscle movements, such as facial expressions or other types of muscle activity, in human subjects. The invention is suitable for use in electronic entertainment or other platforms in which electroencephalograph (EEG) data is collected and analysed in order to determine a subject's facial expression in real-time in order to provide control signals to that platform, and it will be convenient to describe the invention in relation to that exemplary, non-limiting application.

### BACKGROUND

Facial expression has long been one of the most important aspects of human to human communication. Humans have become accustomed to consciously and unconsciously showing our feelings and attitudes using facial expressions. Furthermore, we have become highly skilled at reading and interpreting facial expressions of others. Facial expressions form a very powerful part of our everyday life, everyday communications and interactions.

As technology progresses, more of our communication is mediated by machines. People now “congregate” in virtual chat rooms to discuss issues with other people. Text messaging is becoming more popular, resulting in new orthographic systems being developed in order to cope with this unhuman world. Currently, facial expressions have not been used in man machine communication interfaces. Interactions with machines are restricted to the use of cumbersome input devices such as keyboards and joysticks. This limits our communication to only premeditated and conscious actions.

There therefore exists a need to provide technology that simplifies man-machine communications. It would moreover be desirable for this technology to be robust, powerful and adaptable to a number of platforms and environments. It would also be desirable for this technology to optimise the use of natural human to human interaction techniques so that the man-machine interface is as natural as possible for a human user.

### SUMMARY

With this in mind, one aspect of the invention provides a method of detecting and classifying facial muscle movements including, the steps of:

detecting bio-signals from at least one bio-signal detector; and

applying at least one facial muscle movement-detection algorithm to a portion of the bio-signals affected by a predefined type of facial muscle movement in order to detect facial muscle movements of that predefined type.

The step of applying at least one facial movement-detection algorithm to the bio-signals may include:

comparing the bio-signal portion to a signature defining one or more distinctive signal characteristics of the predefined facial muscle movement type.

In a first embodiment of the invention, the step of applying at least one facial muscle movement-detection algorithm to the bio-signals may include:

directly comparing bio-signals from one or more predetermined bio-signal detectors to the signature.

In another embodiment of the invention, the step of applying at least one facial muscle movement-detection algorithm to the bio-signals may include:

projecting bio-signals from a plurality of bio-signal detectors onto one or more predetermined component vectors; and comparing the projections onto the one or more component vectors to that signature.

The predetermined component vectors may be determined from applying a first component analysis to historically collected bio-signals generated during facial muscle movements of the type corresponding to that first signature. “The first component analysis applied to the historically collected bio-signals may be independent component analysis (ICA). Alternatively, the first component analysis applied to the historically collected bio-signals may be principal component analysis (PCA). ” In this embodiment, the method may further include the steps of:

applying a second component analysis to the detected bio-signals; and

using the results of the second component analysis to update the one or more predetermined component vectors during bio-signal detection.

The second component analysis may be principal component analysis (PCA).

In yet another embodiment of the invention, the step of applying at least one facial muscle movement-detection algorithm to the bio-signals may include:

applying a desired transform to the bio-signals; and

comparing the results of the desired transform to that signature.

The desired transform may be selected from any one or more of a Fourier transform, wavelet transform or other signal transformation method.

The step of applying at least one facial muscle movement-detection algorithm to the bio-signals may further include the step of: separating the bio-signals resulting from the predefined type of facial muscle movement from one or more sources of noise in the bio-signals.

The sources of noise may include any one or more of electromagnetic interference (EMI), bio-signals not resulting from the predefined type of facial muscle movement and other muscle artefacts.

The facial muscle movement types may include facial expressions, such as blinking, winking, frowning, smiling and laughing.

The facial muscle movement may further include other muscle activity, such as eye movements, yawning, chewing and talking.

In one or more embodiments of the invention, the bio-signals may include electroencephalograph (EEG) signals.

The method may further include the step of:

generating a control signal representative of the detected facial muscle movement type for input to an electronic entertainment application or other application.

Another aspect of the invention provides an apparatus for detecting and classifying facial muscle movements including:

a sensor interface for receiving bio-signals from at least one bio-signal detector; and

a processing system for carrying out the step of:

applying at least one facial muscle movement-detection algorithm to a portion of the bio-signals affected by a predefined type of facial muscle movement in order to detect facial muscle movements of that predefined type.

### FIGURES

These and other features, aspects and advantages of the present invention will become better understood with regard to the following description, appended claims, and accompanying figures which depict various views and embodiments of the device, and some of the steps in certain embodiments of the method of the present invention, where:

FIG. 1 is a schematic diagram of an apparatus for detecting and classifying facial muscle movements in accordance with the present invention;

FIG. 2 is a schematic diagram illustrating the positioning of scalp electrodes forming part of a head set used in the apparatus shown in FIG. 1;

FIG. 3 is a flow chart illustrating the broad functional steps performed by the apparatus in FIG. 1.

FIGS. 4 and 5 represent exemplary signals from selected electrodes shown in FIG. 2 during predefined facial movements;

FIG. 6 is a representation of signals from the scalp electrode shown in FIG. 2 during a number of facial muscle movements;

FIG. 7 is a flow chart illustrating the steps performed in the development of signatures defining distinctive signal characteristics of predefined facial muscle movement types used in the apparatus of FIG. 1 during the detection and classification of facial muscle movement;

FIG. 8 is a conceptual representation of the decomposition of signals from the sensors shown in FIG. 2 into predetermined components as performed by the apparatus of FIG. 1, in at least one mode of operation;

FIG. 9 is a representation of a signal from one of the sensors shown in FIG. 2 during a sequence of eye blinks;

FIG. 10 is a flow chart illustrating the steps performed by the apparatus of FIG. 1 both before and during bio-signal detection and classification in at least one mode of operation;

FIG. 11 is a schematic diagram showing an eye blink component vector present in the bio-signals captured from the sensors shown in FIG. 2 during an exemplary eye blink;

FIG. 12 is a flow chart of one exemplary algorithm for detecting and classifying facial muscle movements as eye blinks;

FIG. 13 shows a representation of a bio-signal detected from an exemplary sensor shown in FIG. 2 and subsequent analysis performed on that bio-signal; and

FIG. 14 represents a bio-signal detected from a sensor shown in FIG. 2 and the result of subsequent manipulations performed to that signal over an extended time period.

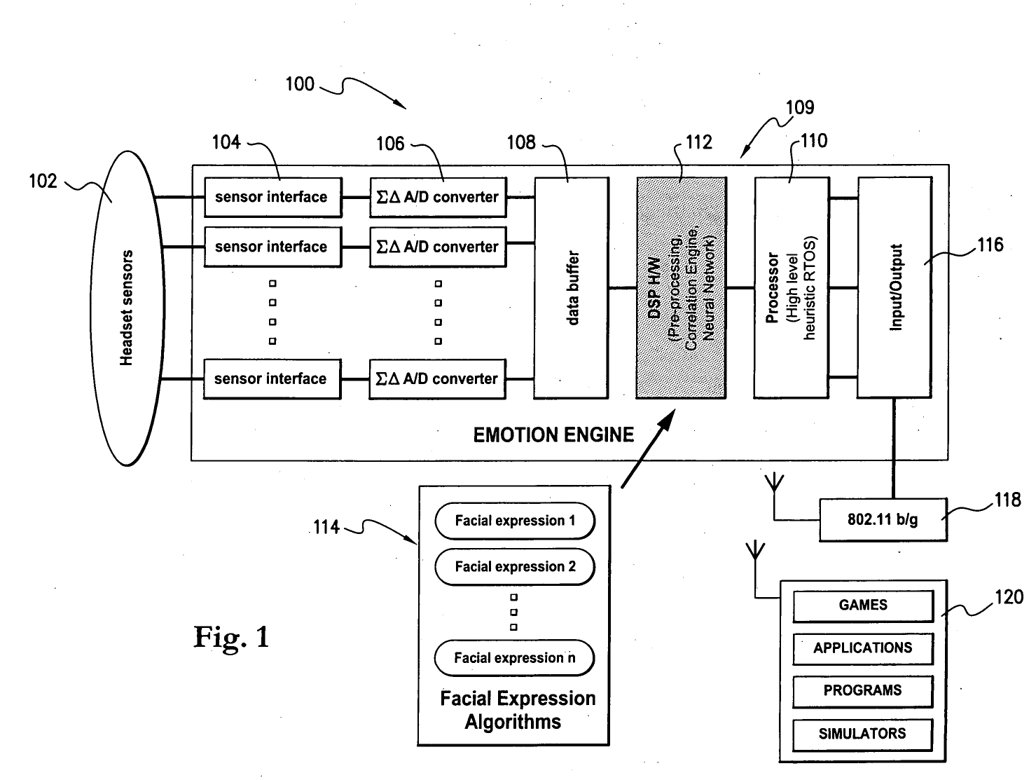

Turning now to FIG. 1, there is shown generally an apparatus 100 for detecting and classifying facial muscle movements. The apparatus 100 includes a headset 102 of bio-signal detectors capable of detecting various bio-signals from a subject such as electroencephalograph (EEG) signals, electroencephalograph (EOG) signals, skin conductance or like signals. In the exemplary embodiment illustrated in the drawings, the headset 102 includes a series of scalp electrodes for capturing EEG signals from the user. The scalp electrodes may directly contact the scalp or alternately may be of the non-contact type that does not require direct placement on the scalp. The electrical fluctuations detected over the scalp by the series of scalp sensors are attributed largely to brain tissue located at or near the skull. The source is the electrical activity of the cerebral cortex, a significant portion of which lies on the outer surface of the brain below the scalp. The scalp electrodes pick up electrical signals naturally produced by the brain and make it possible to observe electrical impulses across the surface of the brain. Although in this exemplary embodiment the headset 102 includes several scalp electrodes, in other embodiments only one or more scalp electrodes may be used in a headset.

Traditional EEG analysis has focused solely on these signals from the brain. The main applications have been explorative research in which different rhythms (alpha wave, beta wave, etc) have been identified, pathology detection in which onset of dementia or physical injury can be detected, and self improvement devices in which bio-feedback is used to aid in various forms of meditation. Traditional EEG analysis considers signals resulting from facial muscle movement such as eye blinks to be artefacts that mask the real EEG signal desired to be analysed. Various procedures and operations are performed to filter these artefacts out of the EEG signals selected.

The applicants have developed technology that enables the sensing and collecting of electrical signals from the scalp electrodes, and the application signal processing techniques to analyze these signals in order to detect and classify human facial expressions such as blinking, winking, frowning, smiling, laughing, talking etc. The result of this analysis is able to be used by a variety of other applications, including but not being limited to electronic entertainment applications, computer programs and simulators.

Each of signals detected by the headset 102 of electrodes is fed through a sensor interface 104 and then digitized by an analogue-to-digital converter 106. Digitized samples of the signal captured by each of the scalp sensors are stored during operation of the apparatus 100 in a data buffer 108 for subsequent processing.

The apparatus 100 further includes a processing system 109 including a processing device 110 and associated memory device for storing a series of instructions (otherwise known as a computer program or computer control logic) to cause the processing device 110 to perform desired functional steps. Notably, the memory device 112 includes a series of instructions defining at least one algorithm 114 for detecting and classifying a predetermined type of facial muscle movement. Upon detection of each predefined type of facial muscle movement, a corresponding control signal is transmitted to an input/output interface 116 for transmission via a wireless transmission device 118 to a platform 120 for use as a control input by electronic entertainment applications, programs, simulators or the like.

In this embodiment, the invention is implemented in software and the series of instructions is stored in the memory device 112. The series of instructions causes the processing device 110 to perform the functions of the invention as described herein. In another embodiment, the invention is implemented primarily in hardware using, for example, hardware components such as application specific integrated circuits (ASICs). Implementation of the hardware state machine so as to perform the functions described herein will be apparent to persons skilled in the relevant art. In yet another embodiment, the invention is implemented using a combination of software and hardware.

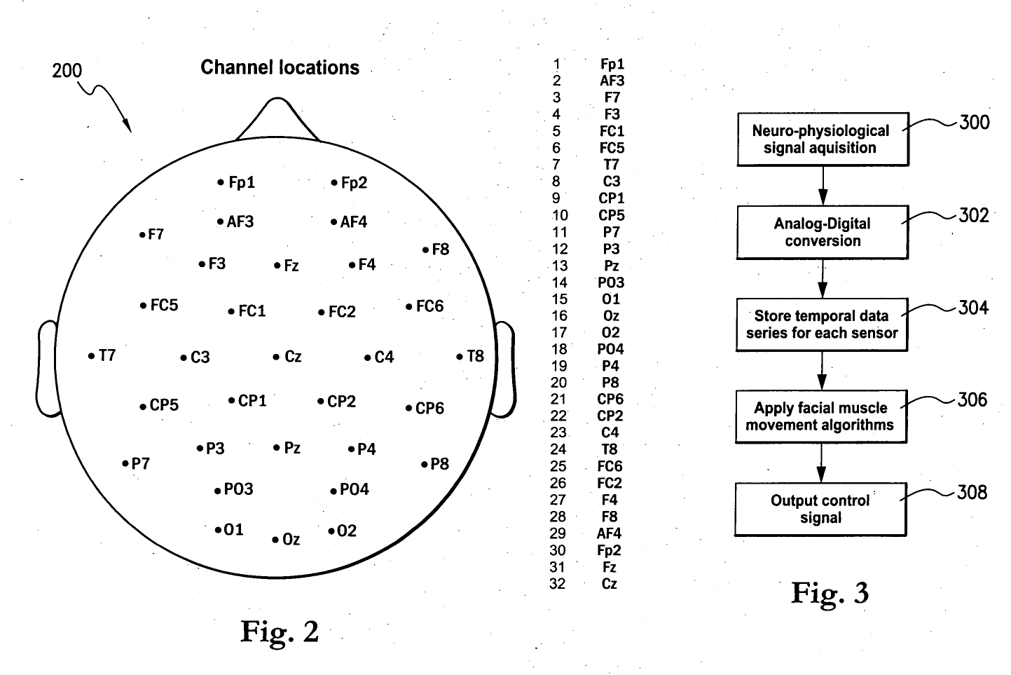

FIG. 2 illustrates one example of the positioning system 200 of the scalp electrodes forming part of the headset 102. The system 200 of electrode placement shown in FIG. 2 is referred to as the “10-20” system and is based on the relationship between the location of an electrode and the underlying area of cerebral cortex. Each point on the electrode placement system 200 indicates a possible scalp electrode position. Each site includes a letter to identify the lobe and a number or other letter to identify the hemisphere location. The letters F, T, C, P, 0 stand for Frontal, Temporal, Central, Parietal and Occipital. Even number refer to the right hemisphere and odd numbers refer to the left hemisphere. The letter Z refers to an electrode placed on the mid-line. The mid-line is a line along the scalp on the sagittal plane originating at the nasion and ending at the inion at the back of the head The “10” and “20” refer to percentages of the mid-line division. The mid-line is divided into 7 positions, namely, Nasion, Fpz, Fz, Cz, Pz, Oz and Inion, and the angular intervals between adjacent positions are 10%, 20%, 20%, 20%, 20% and 10% of the mid-line length respectively.

As seen in FIG. 3, the headset 102 including scalp electrodes positioned according to the system 200 are placed on the head of a subject in order to detect EEG signals. At step 300, the EEG signals are captured by a neuro-physiological signal acquisition device and then converted into the digital domain at step 302 using the analogue to digital converters 106. A series of digitized signals from each of the sensors is then stored at step 304 in the data buffer 108. One or more facial muscle movement-detection algorithms are then applied at step 306 in order to detect and classify different facial muscle movements, including facial expressions or other muscle movements. Each of the algorithms generates a result representing the facial expression(s) of the subject. These results are then passed on to the output block 116 at step 308 where they can be used by a variety of applications.

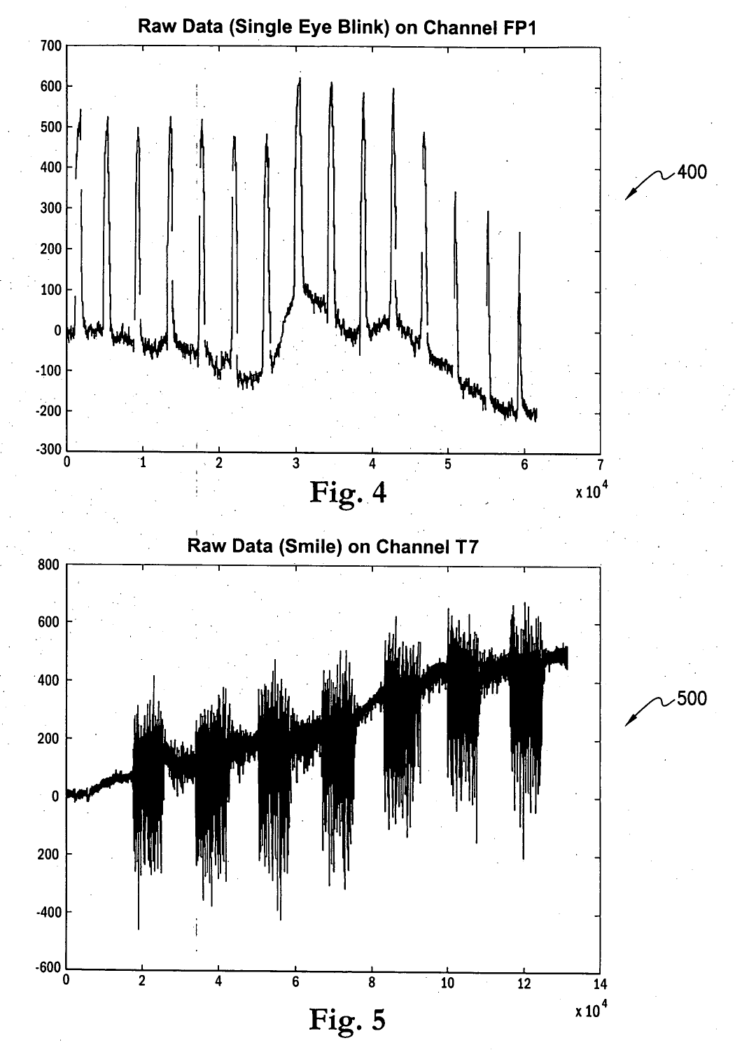

In traditional EEG research, many signals resulting from eye blinks and other facial muscle movements have been considered to be artefacts masking the real EEG signal required for analysis. FIG. 4 shows a representation 400 of a signal from the Fp1 or Fp2 electrode (as seen in the electrode positioning system 200 shown in FIG. 2) during a series of eye blinks. Similarly, FIG. 5 shows a representation 500 of a signal from the T7 or T8 electrode resulting from a series of smiles by a subject.

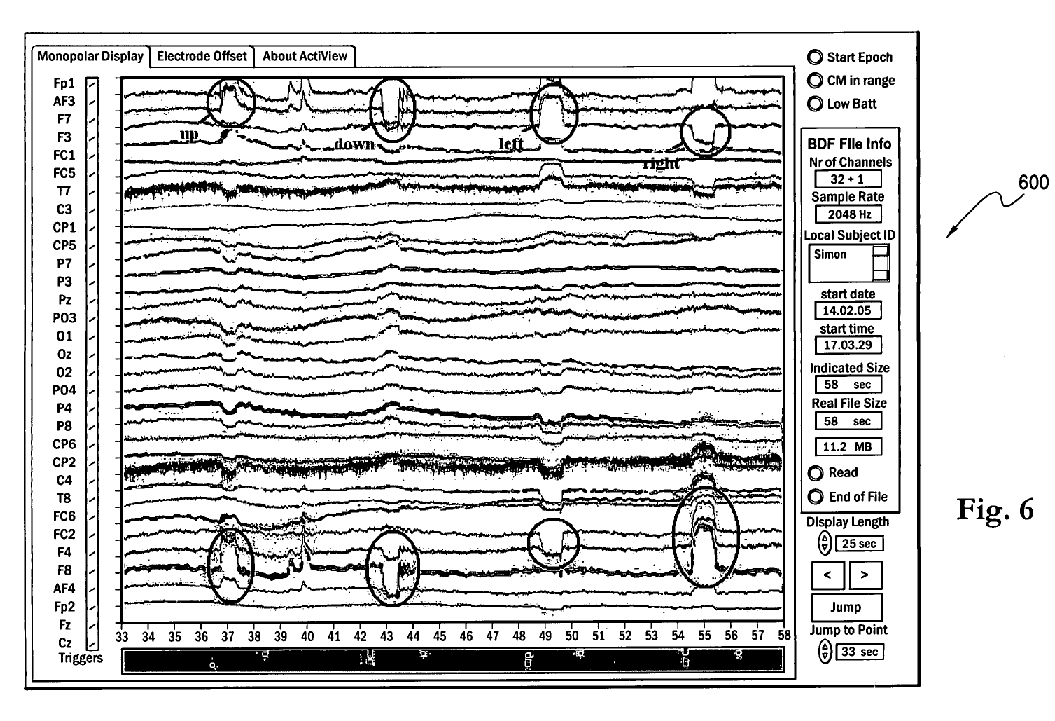

FIG. 6 shows a representation 600 of the signals from each of the electrodes in the headset 102 when various eye movements are performed by the subject. The impact of an up, down, left and right eye movement can be observed from the circle portions of signal representations. Rather than considering the impact upon the EEG signals resulting from facial muscle movements to be an artefact that pollutes the quality of the EEG signals, the apparatus 100 acts to isolate these perturbations and then apply one or more algorithms in order to classify the type of facial muscle movement responsible for producing the perturbations.

The apparatus 100 applies at least one facial muscle movement-detection algorithm 114 to a portion of the bio-signals captured by the headset 102 affected by a predefined type of facial muscle movement in order to detect facial muscle movements of that predefined type. In order to do so, a mathematical signature defining one or more distinctive characteristics of the predefined facial muscle movement type is stored in the memory device 112. The relevant portion of the bio-signals affected by the predefined type of facial muscle movement is then compared to that mathematical signature.

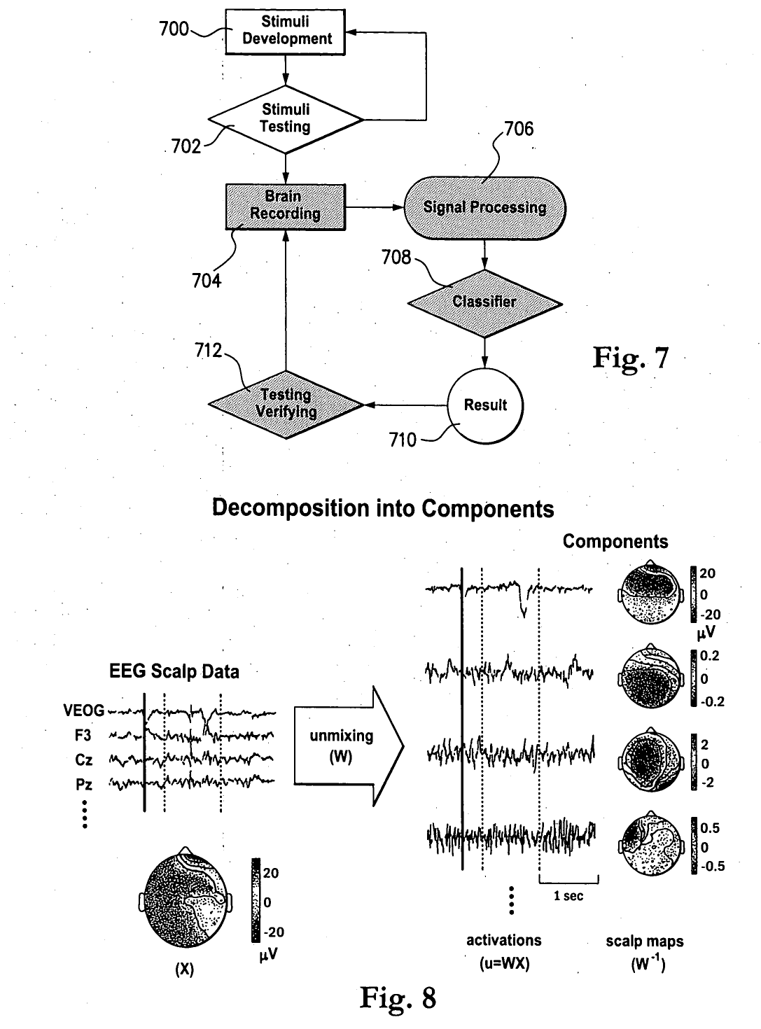

In order to generate the mathematical signature for each facial muscle movement, and as shown in FIG. 7, stimuli are developed at step 700 to elicit that particular facial expression. The stimuli are generally in the form of an audio visual presentation or a set of commands. The set of stimuli is tested as step 702 until a high degree of correlation between the developed stimuli and the resultant desired facial muscle movement is obtained. Once a set of effective stimuli is developed, EEG signal recordings are made at step 704 that contain many examples of the desired facial muscle movements. Ideally, these facial muscle movements should be as natural as possible.

Once the EEG signal recordings are collected, signal processing operations are performed at step 706 in order to identify one or more distinctive signal characteristics of each predefined facial muscle movement type. Identification of these distinctive signal characteristics in each EEG signal recording enables classification of the facial muscle movement in a subject to be classified at step 708 and an output signal representative of the detected type of facial muscle movement output at step 710. Testing and verification of the output signal at step 712 enables a robust data set to be established.

In one of the modes of operation, the portion of the bio-signals affected by a predefined type of facial muscle movement is predominantly found in signals from a limited number of scalp electrodes. For example, eye movement and blinking can be detected by using only two electrodes near the eyes, such as the Fp1 and Fp2 channels shown in FIG. 2. In this case, signals from those sensors can be directly compared to the mathematical signatures defining the distinctive signal characteristics of the eye blink or other predefined facial muscle movement type.

It is also possible to combine the signals from one or more electrodes together, and then to compare that combined bio-signal to a signature defining the distinctive signal characteristics of the predefined facial muscle movement type. A weighting may be applied to each signal prior to the signal combining operation in order to improve the accuracy of the facial muscle movement detection and classification.

In other modes of operation, the apparatus 100 acts to decompose the scalp electrode signals into a series of components and then to compare the projection of the bio-signals from the scalp electrodes onto one or more predetermined component vectors with the mathematical signatures defining the signal characteristics of each type of facial muscle movement. In this regard, independent component analysis (ICA) has been found to be useful for defining the characteristic forms of the potential function across the entire scalp. Independent component analysis maximizes the degree of statistical independence among outputs using a series of contrast functions.

As seen in FIG. 8, in independent component analysis, the rows of an input matrix X represent data samples from the bio-signals in the headset 102 recorded at different electrodes whereas the columns are measurements recorded at different type points. Independent component analysis finds an “unmixing” matrix W which decomposes or linearly unmixes the multi-channel scalp data into a sum of temporarily independent and specially fixed components. The rows of the output data matrix U=WX are time courses of activation of the ICA components. The columns of the inverse matrix, W−1, give the relative projection strength of each of the signals from the scalp electrodes onto respective component vectors. These scalp weights give the scalp topography of each component vector.

Another technique for the decomposition of the bio-signals into components is principal component analysis (PCA) which ensures that output pairs are uncorrelated. In various embodiments of the invention, either or both of independent component analysis and principal component analysis may be used in order to detect and classify facial muscle movements.

In other modes of operation, the apparatus 100 may act to apply a desired Fourier transform to the bio-signals from the scalp electrodes. The transform could alternatively be a wavelet transform or any other suitably signal transformation method. Combinations of one or more different signal transformation methods may also be used. Portions of the bio-signals affected by a predefined type of facial muscle movement may then be identified using a neural network.

Each of the above described techniques for detection and classification of the facial muscle movements may be incorporated into a facial expression algorithm stored in the memory storage device 112. Once a particular facial expression detection algorithm has been fully developed, the algorithm may be implemented as a piece of-real-time software program or transferred into a digital signal processing environment.

As an example of the type of facial muscle movement that can be detected and classified by the apparatus 100, a facial expression algorithm for the detection of an eye blink will now be described. It is to be understood that the general principles described in relation to the algorithm are also applicable to the detection and classification of other types of facial muscle movement.

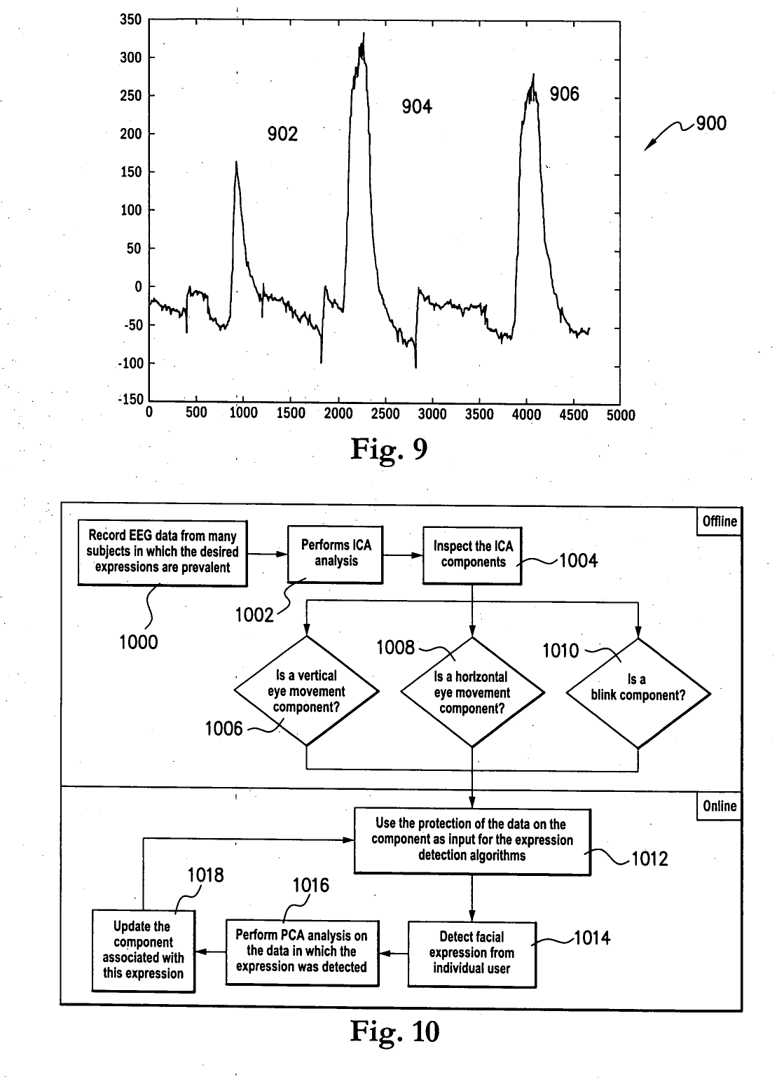

Eye blinks are present in all interior electrodes but feature most prominently in the two frontal channels Fp1 and Fp2. FIG. 9 is a representation 900 of the bio-signal recorded at the scalp electrode Fp1 during 3 typical eye blinks. It can be seen from signal portions 902, 904 and 906 of the bio-signal from the frontal channel Fp1 that each of the 3 eye blinks has a significant effect on the bio-signal. In this example, the projections of the bio-signals from the frontal electrodes Fp1 and Fp2 on predetermined component vectors str used to detect and classify the perturbation in the bio-signals as an eye blink.

In a preferred embodiment of the invention, the predetermined component vectors are identified from historically collected data from a number of subjects and/or across a number of different sessions. As shown in FIG. 10 the EEG data from a number of different subjects and/or across a number of different sessions are recorded at step 1000 when the desired facial muscle movements are being generated by the subjects.

At step 1002, independent component analysis is performed on the recorded EEG data and the component vectors onto which are projected the perturbations in the EEG signals resulting from the relevant facial muscle movement are determined at step 1004. The relevant component vectors to be used in subsequent real-time data recording and analysis are then recorded in the storage device 112 by facial muscle movement type. In this case, three exemplary types of facial muscle movement are able to be classified, namely vertical eye movement at step 1006, horizontal eye movement at step 1008 and an eye blink at step 1010.

However, independent component analysis is a computationally time consuming activity and in many instances is inappropriate for real-time use. Whilst independent component analysis may be used to generate average component vectors for use in the detection and classification of various types of facial muscle movements, the balance of signals across different electrodes vary slightly across different sessions and users.

Accordingly, the average component vectors defined using independent component analysis of historically gathered data will not be optimal during real-time data detection and classification. During real-time operation of the apparatus 100, principal component analysis can be performed on the real-time data and the resulting component vector can be used to update the component vector generated by independent component analysis throughout each session. In this way, the resulting facial muscle movement-detection algorithms can be made robust against electrodes shifting and variances in the strengths of the contacts.

As can be seen at step 1012, the projection of the historically collected data on the vector component is initially used for the facial muscle movement algorithms 114. However, as data is collected and stored in the data buffer 108 at step 1014, principal component analysis is carried out at step 1016 on the stored data, and the results of the analysis generated at step 1018 are then used to update the component vectors developed during offline independent component analysis.

One or more of the component vectors may be updated during facial muscle movement detection and classification in order to improve the accuracy and viability of the facial muscle movement detection algorithms.

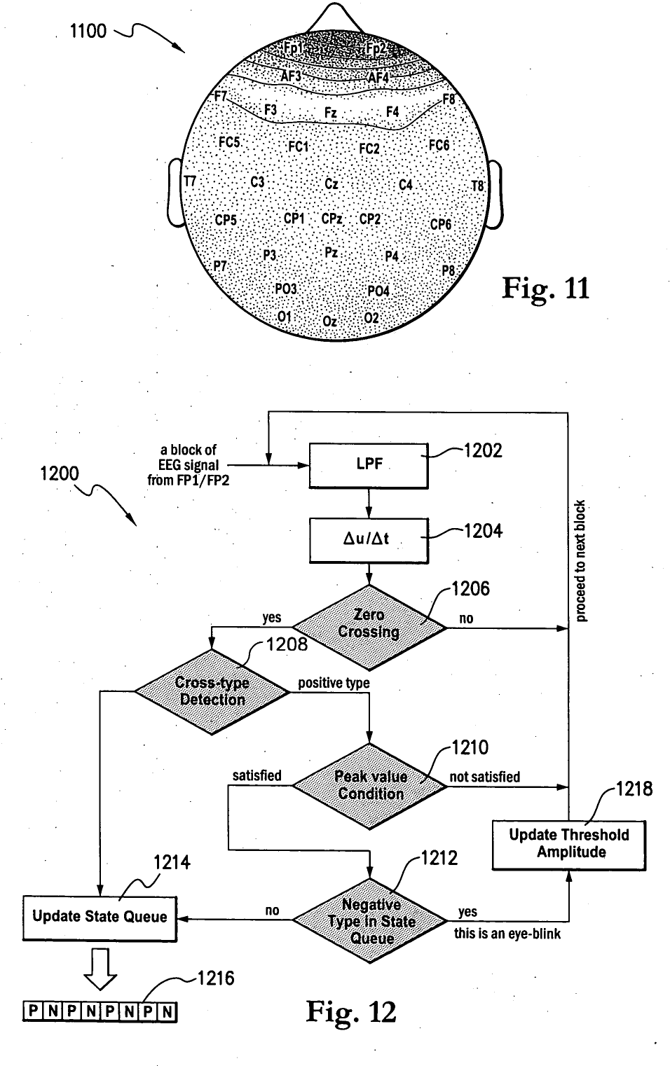

As has been previously described, component vectors can be used in order that a correct weighting is applied to the contribution from the signals of each relevant electrode. An example of an eye-blink component vector is shown in the vector diagram 1100 in FIG. 11. From this diagram it can be seen that the largest contribution to the component is indeed from the two frontal electrodes Fp1 and Fp2. However, it is also apparent that the eye blink is not symmetric. In this case, the potential around the electrode Fp2 is larger than that as the electrode Fp1. The difference may be due to a number of causes, for example, muscle asymmetry, the electrodes not being symmetrically located on the head of a subject or a difference in the electrical impedance contact with the scalp. This diagram illustrates the desirability of optimizing the component vectors during each session, for example by applying the steps illustrated in FIG. 10.

FIG. 12 shows one example of a facial muscle movement-detection algorithm 1200 used to detect an eye blink. The algorithm 1200 may be applied to the activations of component vectors or alternatively may be applied to signals from individual scalp electrodes. In a preferred embodiment the projection of the EEG signals onto the component vector associated with an eye blink is initially passed through a low pass filter at step 1202. A first order derivative operation is then performed on the signal. In short, the first order derivative of a function ƒ with respect to an infinitesimal change χ is defined as

$${f}^{1} ( x ) = {lim}_{h \to 0} \frac{f ( x + h ) - f ( x )}{h}$$

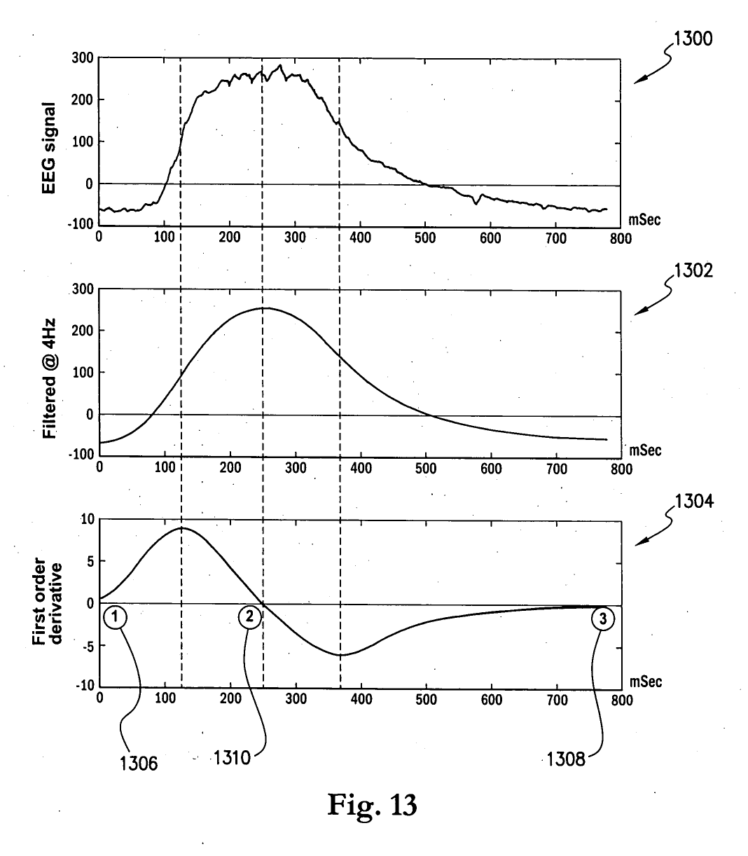

and it represents an infinitesimal change in the function with respect to χ. For eye blink detection, a derivative of the signal with respect to time is taken at step 1204 the result of low pass filtering and the first order derivative operation on the component vector for an eye blink is shown in FIG. 13. The original component vector is referenced 1300, whereas the signal resulting from the low pass filtering, and from the first order derivative operation are referenced 1302 and 1304 respectively.

Of particular interest are zero-crossing points in the first order derivative signal, which fall into two categories: positive zero-crossing point and negative zero-crossing point. The sign (namely either positive or negative) of the zero-crossing points indicates whether the signal increases or decreases after crossing the axis. For each eye blink, there are two positive zero-crossing points, respectively referenced 1306 and 1308 on FIG. 13. These positive zero-crossing points define boundary conditions of an eye blink. A negative zero-crossing point 1310 defines the peak of the eye blink. Accordingly, the algorithm 1200 determines at step 1206 whether a zero-crossing point occurs in the digitized data stored in data buffer 108. If this is the case, a determination is made a step 1208 if the crossing type was a positive or a negative zero-crossing. If a positive crossing was detected, its peak amplitude is checked at step 1210 to verify whether this positive zero-crossing is from a real eyeblink. If the positive zero-crossing point satisfies peak value condition, the algorithm stores this information into state queue at step 1214 in cases where there is no preceding negative zero-crossing point determined at step 1212 to be stored in the queue. If there is a preceding negative zero-crossing point stored in the state queue, an assertion that there is an eyeblink is made at step 1212. The algorithm resets if there is no zero-crossing point found; or found zero-crossing point does not satisfy peak value condition; or an eyeblink detection assertion is made.

Accordingly, once the zero-crossing points are identified, the algorithm verifies whether there exists a negative zero-crossing point sandwiched between the two positive zero-crossing points, and the eye blink peak passes amplitude threshold. A default value of the amplitude threshold is initially made, but to increase the accuracy of the algorithm, the threshold amplitude is adjusted at step 1218 based upon the strength of an individuals eyes blink peaks.

In this example, the eye blink “signature” defines the distinctive signal characteristics representative of an eye blink, namely a negative zero crossing sandwiched between two positive zero crossings in the first order derivative of the filtered signal, and a signal amplitude greater than a predetermined threshold in the filtered signal. The signature is updated by changing the threshold forming part of the distinctive signal characteristics of the signature during facial muscle movement detection and classification. In other embodiments, the digital signature may define other amplitudes or signal characteristics that exceed one or more predetermined thresholds. The signature may be updated during facial muscle movement detection and classification by changing one or more of those thresholds. More generally, any one or more distinctive signal characteristics of a predetermined facial muscle movement type that form part of a digital signature can be updated during the course of facial muscle movement detection and classification in order to improve the viability and accuracy of the facial muscle movement detection algorithms implemented by the apparatus 100.

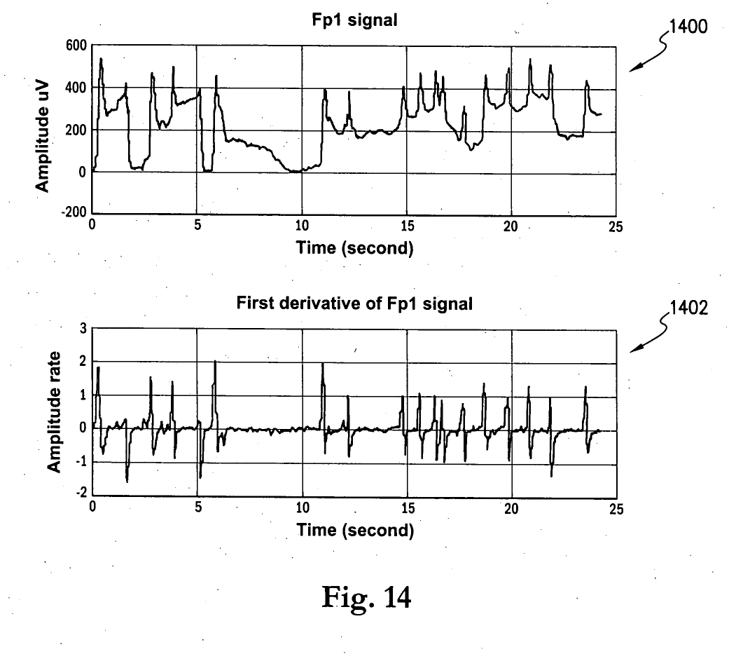

The result of applying the above described operations to an EEG signal recorded at, for example, the electrode Fp1 containing eye blinks is shown in FIG. 14. The first representation referenced 1400 shows the unprocessed signal, whereas the second representation referenced 1402 shows the first order derivative signal over an expanded time frame.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

What is claimed is:

1. A method of detecting and classifying facial muscle movements, comprising the steps of:
a) detecting bio-signals from one or more than one bio-signal detector; and
b) applying one or more than one facial muscle movement-detection algorithm to a portion of the bio-signals affected by a predefined type of facial muscle movement in order to detect the facial muscle movements of the predefined type.

2. The method according to claim 1, wherein the step of applying one or more than one facial muscle movement-detection algorithm to the bio-signals comprises comparing the bio-signal portion to a signature defining one or more than one distinctive signal characteristics of the predefined facial muscle movement type.

3. The method according to claim 2, wherein the step of applying one or more than one facial muscle movement-detection algorithm to the bio-signals comprises directly comparing bio-signals from one or more than one predetermined bio-signal detectors to that signature.

4. The method according to claim 2, wherein the step of applying one or more than one facial muscle movement-detection algorithm to the bio-signals comprises:
a) projecting bio-signals from the plurality of bio-signal detectors on one or more than one predetermined component vectors; and
b) comparing the projection of the bio-signals onto one or more than one component vectors to that signature.

5. The method according to claim 4, further comprising applying a desired transform to the projected bio-signal after the projection of the bio-signals from the plurality of detectors on one or more than one component vectors, and before the projected bio-signal is compared to that signature.

6. The method according to claim 4, wherein the predetermined component vectors are determined from applying a first component analysis to historically collected bio-signals generated during facial muscle movement types of the type corresponding to that signature.

7. The method according to claim 6, wherein the first component analysis applied to the historically collected bio-signals is independent component analysis (ICA).

8. The method according to claim 6, wherein the first component analysis applied to the historically collected bio-signals is principal component analysis (PCA).

9. The method according to claim 4, wherein the one or more than one component vectors are updated during facial muscle movement-detection and classification.

10. The method according to claim 2, further comprising updating the signature during the course of facial muscle movement-detection and classification.

11. The method according to claim 10, wherein the signature is updated by changing thresholds forming at least part of the distinctive signal characteristics of the signature.

12. The method according to claim 2, wherein the step of applying one or more than one facial muscle movement-detection algorithm to the bio-signals comprises:
a) applying a desired transform to the bio-signals; and
b) comparing the results of the desired transform to that signature.

13. The method according to claims 12, wherein the transform is one or more than one transform selected from the group consisting of a Fourier transform and a wavelet transform.

14. A method according to claim 4 further comprising:
a) applying a second component analysis to the detected bio-signals; and
b) using the results of the second component analysis to update the one or more than one predetermined component vectors during bio-signal detection.

15. The method according to claim 14, wherein the second component analysis is principal component analysis (PCA).

16. The method according to claim 1, wherein the step of applying one or more than one facial muscle movement-detection algorithm to the bio-signals comprises separating the bio-signals resulting from the predefined type of facial muscle movement from one or more than one sources of noise in the bio-signals.

17. The method according to claim 16, wherein the sources of noise comprise one or more than one source selected from the group consisting of electromagnetic interference (EMI), and bio-signals not resulting from the predefined type of facial muscle movement.

18. The method according to claim 1, wherein the facial muscle movement type is one or more than one facial muscle movement type selected from the group consisting of blinking, winking, frowning, smiling and laughing.

19. The method according to claim 1, wherein the facial muscle movement type is one or more than one facial muscle movement type selected from the group consisting of eye-movements, yawning, chewing and talking.

20. The method according to claim 1, wherein the bio-signals comprise electroencephalograph (EEG) signals.

21. The method according to claim 1, further comprising generating a control signal representative of the detected facial muscle movement type for input to a gaming application.

22. An apparatus for detecting and classifying facial muscle movements, comprising:
a) a sensor interface for receiving bio-signals from one or more than one bio-signal detector; and
b) a processing system for carrying out the step of applying one or more than one facial muscle movement-detection algorithm to a portion of the bio-signals affected by a predefined type of facial muscle movement in order to detect facial muscle movements of that predefined type.

23. The apparatus according to claim 22, wherein the processing system compares the bio-signal portion to a signature defining one or more than one distinctive signal characteristics of the predefined facial muscle movement type.

24. The apparatus according to claim 23, wherein the processing system directly compares bio-signals from one or more than one predetermined bio-signal detectors to that signature.

25. The apparatus according to claim 23, wherein the processing system projects bio-signals from the plurality of bio-signal detectors on one or more than one predetermined component vectors; and then compares the projection of the bio-signals onto one or more than one component vectors to that signature.

26. The apparatus according to claim 25, wherein after the projection of the bio-signals from the plurality of detectors on one or more than one component vectors and before the projected bio-signal is compared to that signature; a desired transform is applied to the projected bio-signal.

27. The apparatus according to claim 25, wherein the predetermined component vectors are determined from applying a first component analysis to historically collected bio-signals generated during facial muscle movement types of the type corresponding to that signature.

28. The apparatus according to claim 27, wherein the first component analysis applied to the historically collected bio-signals is independent component analysis (ICA).

29. The apparatus according to claim 27, wherein the first component analysis applied to the historically collected bio-signals is principal component analysis (PCA).

30. The apparatus according to claim 25, wherein the one or more than one component vectors are updated during facial muscle movement-detection and classification.

31. The apparatus according to claim 23, wherein the signature is updated during the course of facial muscle movement-detection and classification.

32. The apparatus according to claim 31, wherein the signature is updated by changing thresholds forming at least part of the distinctive signal characteristics of the signature.

33. The apparatus according to claim 23, wherein the processing system applies a desired transform to the bio-signals; and compares the results of the desired transform to that signature.

34. The apparatus according to claims 33, wherein the transform is selected from one or more than one transform selected from the group consisting of a Fourier transform and a wavelet transform.

35. A method according to claim 25, wherein the processing system applies a second component analysis to the detected bio-signals, and uses the results of the second component analysis to update the one or more than one predetermined component vectors during bio-signal detection.

36. The apparatus according to claim 35, wherein the second component analysis is principal component analysis (PCA).

37. The apparatus according to claim 22, wherein the processing system separates the bio-signals resulting from the predefined type of facial muscle movement from one or more than one sources of noise in the bio-signals.

38. The apparatus according to claim 37, wherein the sources of noise comprise one or more than one selected from the group consisting of electromagnetic interference (EMI), and bio-signals not resulting from the predefined type of facial muscle movement.

39. The apparatus according to claim 22, wherein the facial muscle movement types comprise one or more than one facial expression selected from the group consisting of blinking, winking, frowning, smiling and laughing.

40. The apparatus according to claim 22, wherein facial muscle movement types comprise one or more than one facial expression selected from the group consisting of eye-movements, yawning, chewing and talking.

41. The apparatus according to claim 22, wherein the bio-signals comprise electroencephalograph (EEG) signals.

42. The apparatus according to claim 22, wherein the processing system generates a control signal representative of the detected facial muscle movement type for input to a gaming application.
